(19) United States
(12) Patent Application Publication
(10) Pub. No.: US 2026/0284126 A1
(43) Pub. Date: Sep. 24, 2026

(54) LACTOBACILLUS COMPOSITIONS FOR TREATING TYPE 2 DIABETES

(71) Applicant: UNIVERSITY OF FLORIDA RESEARCH FOUNDATION, INCORPORATED, Gainesville, FL (US)

(72) Inventors: Graciela Liliana LORCA, Gainesville, FL (US); Claudio F. GONZALEZ, Gainesville, FL (US)

(21) Appl. No.: 19/475,256

(22) PCT Filed: Apr. 15, 2024
(86) PCT No.: PCT/US2024/024624

§ 371 Date: Oct. 14, 2025

Related U.S. Application Data
(60) Provisional application No. 63/459,342, filed on Apr. 14, 2023.

(51) Int. Cl. A61K 35/747; A61K 9/48; A61P 1/16; A61P 3/10; A61P 13/12

(52) U.S. Cl. CPC A61K 35/747; A61K 9/4816; A61K 9/4858; A61K 9/4875; A61P 1/16; A61P 3/10; A61P 13/12

(57) ABSTRACT

It is disclosed herein that administration of Lactobacillus johnsonii N6.2 has a remarkable beneficial effect on diabetes symptoms in DB/DB mice. Based on these results, provided are methods of treat type 2 diabetes involving the administration of L. johnsonii N6.2. In addition, methods of treating or ameliorating steatosis and diabetic nephropathy are disclosed.

### BACKGROUND

Type-2 diabetes refers to a condition in which insulin, a glucose-regulating hormone secreted from beta cells of the pancreas, cannot be produced in an amount required in the body, or insulin does not act properly on cells, and thus glucose in the blood is accumulated in the blood without being used as energy to induce hyperglycemia, and glucose is detected in urine. Type 2 diabetes develops because the insulin action is insufficient or insulin is relatively insufficient due to insulin resistance, and 90% of diabetic patients belong to type 2 diabetes, and type 2 diabetes is called adult-type diabetes because it usually develops in adults over thirty.

If diabetes persists for a long time, the absorption of glucose in the body does not occur normally, resulting in abnormal carbohydrate metabolism, lipid metabolism, and protein metabolism, so that various diabetic complications such as hyperinsulinemia, neurological complications, diabetic retinopathy (non-proliferative retinopathy, proliferative retinopathy, and diabetic cataracts), renal failure, sexual dysfunction, skin disease (allergy), hypertension, arteriosclerosis, stroke (apoplexy), heart disease (myocardial infarction, angina pectoris, and cardioplegia), and gangrene develop. Accordingly, studies on glucose transport and metabolic processes, the insulin signaling system, and the like have been actively conducted at home and abroad in order to understand the various causes and pathogenesis of type 2 diabetes and make improvement measures, but a drug capable of treating type 2 diabetes fundamentally has not yet been developed.

Type 1 diabetes has a different etiology from Type 2 diabetes. Evidence is increasing available demonstrating that Type 1 diabetes is the result of an autoimmune response whereby the body's immune system attacks insulin producing cells. Studies have also demonstrated that the immune response associated with Type-1 diabetes can be ameliorated by manipulating a subject's gut microbiome, and in particular, by administering a certain strain of enterobacteria, Lactobacillus johnsonii N6.2 (U.S. Pat. No. 10,925,907).

### BRIEF DESCRIPTION OF THE DRAWINGS

FIG. 1 is a diagram of a study design related to embodiments.

FIG. 2A provides a graph showing the effects of L. johnsonii N6.2 on fasting blood glucose.

FIG. 2B provides a graph showing the effects of L. johnsonii N6.2 on blood glucose.

FIG. 2C provides a graph showing the effects of L. johnsonii N6.2 on insulitis scores.

FIG. 3 provides a graph showing the effects of L. johnsonii N6.2 on steatosis scores.

FIG. 4A provides a graph showing the effects of L. johnsonii N6.2 on white adipose cell counts.

FIG. 4B provides a graph showing the effects of L. johnsonii N6.2 on white adipose cell size.

FIG. 4C provides a graph showing the effects of L. johnsonii N6.2 on white adipose cell diameter.

FIG. 5 provides a graph showing the effects of L. johnsonii N6.2 on mesangial matrix.

FIG. 6: provides a results summary of the global mRNA analyses performed in liver tissue upon administration of L. johnsonii N6.2. The identity of genes that were significantly modified in liver tissue is indicated in the volcano plot.

### DETAILED DESCRIPTION

In accordance with the subject invention, it has been found that Lactobacillus strains can be used to alleviate (delay the onset of, and/or reduce the severity or progression of), type 2 diabetes (T2D). In specific embodiments of the subject invention, the administration of Lactobacillus strains such as L. johnsonii can prevent or delay the onset of, or reduce the progression of, T2D.

In one embodiment, isolated Lactobacillus strains are used in methods to delay and/or prevent the development of T2D. The preferred strains include Lactobacillus reuteri and Lactobacillus johnsonii. In one embodiment the bacteria is L. johnsonii. Specially exemplified herein is Lactobacillus johnsonii N6.2.

In certain embodiments, provided is a method for treating type 2 diabetes (T2D) in a subject in need that involves administering to the subject in need an effective amount of a composition comprising a Lactobacillus sp wherein said composition, optionally, further comprises a carrier. The subject in need is in a pre-diabetic state or a diabetic state. In a specific embodiment, the composition pertains to a DR capsule comprising a lyophilized mixture of the Lactobacillus sp and the carrier, wherein the DR capsule is, optionally, comprised of hydrophilic cellulose derivative and an anionic polymer. In addition, the composition may further comprise an antioxidant. In certain embodiments, the Lactobacillus sp is administered to a human subject at a dosage ranging from 10² to 10¹¹ Lactobacillus per day. In a specific embodiment, the composition comprises about 10² to 10¹¹ Lactobacillus johnsonii. In an even more specific embodiment, the method involves orally administering to the subject in need Lactobacillus johnsonii N6.2 having ATCC Accession No. PTA-122064.

According to another embodiment, provided is DR capsule comprising a lyophilized mixture of the Lactobacillus sp and the carrier contained within the DR capsule. The DR capsule may include a shell comprised of hydrophilic cellulose derivative and an anionic polymer. In certain examples, the hydrophilic cellulose derivative is hypromellose and the anionic polymer is selected from polyvinyl acetate phthalate, sodium carboxymethylcellulose, sodium alginate, xanthan gum, Carbopol (cross-linked acrylic acid polymers), cellulose acetate phthalate, hydroxypropyl-methylcellulose phthalate, methacrylic acid copolymer, hydroxypropylmethyl acetate succinate, and mixtures thereof. The DR capsule may further include vitamins, minerals, peptides, enzymes, dried skim milk and coenzymes contained with the DR capsule.

In another embodiment, provided is a method of treating fatty liver disease in a subject in need. The method involves administering to the subject in need an effective amount of a composition comprising a Lactobacillus sp wherein said composition, optionally, further comprises a carrier. In a specific embodiment, the Lactobacillus sp is Lactobacillus johnsonii N6.2 having ATCC Accession No. PTA-122064. The composition may be a DR capsule with a shell containing the Lactobacillus sp.

In yet a further embodiment provided is method of treating diabetic nephropathy in a subject in need. The method involves administering to the subject in need an effective amount of a composition comprising a Lactobacillus sp wherein said composition, optionally, further comprises a carrier. In a specific embodiment, the Lactobacillus sp is Lactobacillus johnsonii N6.2 having ATCC Accession No. PTA-122064. The composition may be a DR capsule with a shell containing the Lactobacillus sp.

### Definitions

The terms “pre-diabetic,” “pre-diabetes,” “pre-diabetic state,” or “pre type-2 diabetes,” as used herein refers to a subject exhibiting a fasting blood glucose level between 100 and 125 mg/dl. The term “diabetic state,” or “diabetes” or “type-2 diabetes” or “T2D” as referred to herein refers to a state in which the fasting blood glucose reaches at least 126 mg/dL. A subject with diabetes or pre-diabetes may exhibit one or more further symptoms including A1C levels of 5.7-6.4% (pre-diabetes) or 6.5% (diabetes), frequent urination, thirst, hunger, fatigue, blurry vision, slow wound healing, and tingling, numbness or pain in hands or feet. Also, pre-diabetes and diabetes is associated with a number of risk factors including obesity, high cholesterol, vascular disease and smoking.

The term “Lactobacillus composition” refers to a composition comprising a Lactobacillus sp. The Lactobacillus composition may include other additives including but not limited to a phenol. Lactobacillus compositions may also include lyophilized versions of constituent(s).

The term “Lactobacillus sp.” refers to Lactobacillus johnsonii and Lactobacillus reuteri. In a specific embodiment, Lactobacillus johnsonii pertains to a specific strain L. johnsonii N6.2. A culture of Lactobacillus johnsonii N6.2 has been deposited with the American Type Culture Collection (ATCC), 10801 University Blvd., Manassas, Va. 20110-2209 USA. The deposit has been assigned accession number ATCC No. PT A-122064 by the repository and was deposited on Mar. 19, 2015. The subject culture has been deposited under conditions that assure that access to the culture will be available during the pendency of this patent application to one determined by the Commissioner of Patents and Trademarks to be entitled thereto under 37 CFR 1.14 and 35 U.S.C. 122. The deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposit will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least 30 (thirty) years after the date of deposit or for the enforceable life of any patent which may issue disclosing the culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it.

The term “isolated” with respect to a Lactobacillus sp refers to Lactobacillus spp. isolated from a natural, heterogeneous environment (e.g., a host) or population of microorganisms and separated via known microbiological techniques from the community of other microorganisms in its environment of origin. The isolated Lactobacillus sp. may then be grown in culture. An isolated Lactobacillus sp. or culture of the isolated Lactobacillus sp is not necessarily free of all possible impurities, but it is a substantially homogenous culture of the bacterial isolate and can be distinguished from a naturally occurring, heterogeneous group of microorganisms.

The term “subject” refers to a human or non-human mammal.

A “subject in need” related to treating diabetes refers to a subject that exhibits one or more symptoms of pre-diabetes or diabetes and/or has one or more risk factors.

The term “co-administration” “co-administer (ed)” or “co-administering” as used herein refers to the administration of an enumerated agent before, concurrently, or after the administration of another enumerated agent such that the biological effects of either agents overlap. The combination of agents as taught herein can act synergistically to treat or prevent the various diseases, disorders or conditions described herein. Using this approach, one may be able to achieve therapeutic efficacy with lower dosages of each agent, thus reducing the potential for adverse side effects.

The term “subject in need” related to treating fatty liver disease refers to a subject that has one or more symptoms or markers of fatty liver disease, behavior profile, or corollary medical conditions, or who has been diagnosed with fatty liver disease.

The term “fatty liver disease” as used herein refers to non-alcoholic fatty liver disease (NAFLD) and alcohol related liver disease (ARLD).

The term “Nonalcoholic fatty liver disease” or “NAFLD” refers to a fatty liver disease characterized by the presence of fat (lipids) in the liver and no substantial inflammation or liver damage. NAFLD can progress into nonalcoholic steatohepatitis and then into irreversible, advanced liver scarring or cirrhosis. NAFLD includes a spectrum of histological forms including hepatic steatosis, and non-alcoholic steatohepatitis (NASH), which is characterized by liver inflammation, steatosis, necrosis and fibrosis due to the disruption of liver cells. NASH resembles alcoholic liver disease, but occurs in people who drink little or no alcohol. The major feature in NASH is fat in the liver, along with inflammation and damage. NASH can lead to cirrhosis, in which the liver is permanently damaged and scarred and is no longer able to function properly. A differential diagnosis of NASH versus NAFLD may be determined by liver biopsy. NAFLD may include nonalcoholic steatohepatitis (NASH) or nonalcoholic cirrhosis.

Individuals with NAFLD may be asymptomatic but clinical lab tests can show elevated liver enzyme levels. Individuals may exhibit symptoms of NAFLD, such as abdominal discomfort (e.g., discomfort in the right upper abdominal quadrant), acanthosis nigricans, bowel dismotility, coma, constipation, disseminated intravascular coagulopathy, epigastric pain, fatigue, malaise, hepatomegaly (generally with a smooth, firm surface upon palpation), hypoglycemia, jaundice, lipomatosis, lipoatrophy, lipodystrophy, nausea, neurological defects, Palmer erythema, panniculitis, periumbilical pain, small bowel bacterial overgrowth, spider angiomata, splenomegaly, subacute liver failure, and vomiting. Clinical evaluation to rule out alcohol related fatty liver disease may include determining if the individual consumes excess alcohol (e.g., >60 g/day for men and >20 g/day for women within the past 5 years. The presence or level of anti-hepatitis C antibody and serum ceruloplasmin levels can be used to look for other causes of liver disease besides NAFLD.

In an individual suspected of having NAFLD or NASH, baseline testing of serum may include measuring or determining levels of AST, ALT, total and direct bilirubin, and fasting serum glucose, as well as a lipid panel. For example, steatosis may be indicated by elevated serum levels (often moderately elevated, e.g., elevated approximately 2, 3, 4, 5, 6, 7, 9, 10, 11, or 12-fold above normal levels) of liver enzymes (such as, e.g., AST, ALT, GGT and alkaline phosphatase) when other causes (such as, e.g., acute hepatitis, autoimmune disease, chronic hepatitis, cirrhosis, fulminant hepatitis, hepatocellular carcinoma, metastatic carcinoma, right heart failure, and viral hepatitis) have been eliminated. For example, ALT values greater than 32, 24, or 56 units per liter of serum or at least 2, 3, 4, 5, 6, 7, 8, 9, 10, 11, 12, or more times normal values may be indicative of a disorder associated with hepatic lipid deposits, or by AST values greater than 40 units per liter of serum or at least 2, 3, 4, 5, 6, 7, 8, 9, 10, 11, 12, or more times normal values. Mild to moderate elevation of serum aminotransferase levels is most commonly found (mean range, 100-200 IU/L). The ratio of AST/ALT is often less than one in NAFLD, but may be greater than one in patients with alcoholic liver disease or advanced liver disease or if the patient advances to fibrosis. GGT levels may also be significantly elevated, e.g., at least 2, 3, 4, 5, 6, 7, 8, 9, 10, 11, 12, or more times normal values as defined by a normal, healthy individual. Liver enzyme levels can be normal in a large percentage of patients with NAFLD, thus normal AST or ALT levels do not exclude the presence of advanced disease. Serum alkaline phosphatase and GGT levels may be mildly abnormal. Given that more than 80% of patients with NAFLD have some components of metabolic syndrome, serum levels of fasting cholesterol and triglycerides, as well as fasting glucose and insulin, may be determined. Albumin, bilirubin, and platelet levels may be normal unless the disease has evolved to cirrhosis. Some patients with NAFLD have low titers of autoimmune antibodies (e.g., antinuclear and anti-smooth muscle antibody) and an elevation of ferritin (Carey et al., “Nonalcoholic Fatty Liver Disease” in Current Clinical Medicine, 2nd edition, Elsevier, New York. In some embodiments, an AST/ALT ratio of greater than 1 can predict more advanced fatty liver disease.

The term “diabetic nephropathy” refers to early diabetic nephropathy and overt diabetic nephropathy.

“Early diabetic nephropathy” is defined as persistent microalbuminuria measured on at least two different occasions as an albumin excretion rate of 30 to 300 mg/24 h or equivalent thereof (for the equivalent thereof, see table below).

“Overt diabetic nephropathy” is defined as albumin excretion rate beyond the microalbuminuric range, i.e. >300 mg/24 h or equivalent thereof (for equivalent thereof, see table below).

Relationship among categories for albuminuria, proteinuria, albumin/creatinine ratio and protein/creatinine ratio:

| | Categories | | |
|---|---|---|---|
| | | Early | Overt |
| | | Diabetic | Diabetic |
| | Normal | Nephropathy | Nephropathy |
| | to mildly | Moderately | Severely |
| Measure | increased | increased | increased |
| Albumin excretion rate | <30 | 30-300 | >300 |
| (AER); (mg/24 h) | | | |
| Protein excretion rate | <150 | 150-500 | >500 |
| (PER); (mg/24 h) | | | |
| Albumnin/creatinine | | | |
| ratio (ACR); | | | |
| (mg/mmol) | <3 | 3-30 | >30 |
| (mg/g) | <30 | 30-300 | >300 |
| Protein/creatinine | | | |
| ratio (PCR); | | | |
| (mg/mmol) | <15 | 15-50 | >50 |
| (mg/g) | <150 | 150-500 | >500 |

Albuminuria and proteinuria can be measured using excretion rates in timed urine collections, ratio of concentrations to creatinine concentration in spot urine samples. Relationships among measurement methods within a category are not exact. The relationships between AER and ACR and between PER and PCR are based on the assumption that average creatinine excretion rate is approximately 1.0 g/24 h or 10 mmol/24 h. The conversions in above table are rounded for pragmatic reasons (for an exact conversion from mg/g of creatinine to mg/mmol of creatinine, multiply by 0.113). Creatinine excretion varies with age, sex, race and diet; therefore the relationship among these categories is approximate only. ACR<10 mg/g (<1 mg/mmol) is considered normal; ACR 10-30 mg/g (1.0-3 mg/mmol) is considered high normal (Kidney International Supplements (2013), Vol 3, Issue 1; KDIGO CKD Work group).

Urine or serum biomarkers for diabetic nephropathy can also be used to determine a patient who has early diabetic nephropathy or overt diabetic nephropathy such as those described in, for example, US Pat. Pub. No. US20100197033; PCT Pub No. WO/2022/044059A1; US Pat Pub. No. US20180313871; and U.S. Patent Pub No. US20170153249.

The term “subject in need” related to treating diabetic nephropathy refers to a subject that has one or more symptoms or markers of diabetic nephropathy disease, or corollary medical conditions, or who has been diagnosed with diabetic nepropathy disease.

As used herein, by the term “effective amount,” “amount effective,” “therapeutically effective amount,” or the like, it is meant an amount effective at dosages and for periods of time necessary to achieve the desired result. These terms refers to an amount of an enumerated agent, which, when administered or co-administered in a proper dosing regimen, is sufficient to reduce or ameliorate the severity, duration, or progression of the disorder being treated (e.g., type 2 diabetes, NAFLD, ARLD, early diabetic nephropathy or over diabetic nephropathy), prevent the advancement of the disorder being treated (e.g., type 2 diabetes, NAFLD, ARLD, early diabetic nephropathy or over diabetic nephropathy), cause the regression of the disorder being treated (e.g., type 2 diabetes, NAFLD, ARLD, early diabetic nephropathy or over diabetic nephropathy), or enhance or improve the prophylactic or therapeutic effects(s) of another therapy. The full therapeutic effect does not necessarily occur by administration of one dose and may occur only after administration of a series of doses. Thus, a therapeutically effective amount may be administered in one or more administrations per day for successive days.

The terms “treat”, “treating” or “treatment of” as used herein refers to providing any type of medical management to a subject. Treating includes, but is not limited to, administering a composition to a subject using any known method, for purposes such as curing, reversing, alleviating, reducing the severity of, inhibiting the progression of, or reducing the likelihood of a disease, disorder, or condition or one or more symptoms or manifestations of a disease, disorder or condition.

As used herein, the term “preventing” means causing the clinical symptoms of the disease state not to develop, e.g., inhibiting the onset of disease, in a subject that may be exposed to or predisposed to the disease state, but does not yet experience or display symptoms of the disease state.

### Compositions

In one aspect, the Lactobacillus sp. bacteria in the disclosed composition may be provided as a dispersion in a solution or media. In another aspect, the Lactobacillus sp bacteria in the disclosed composition may be provided as a semi-solid or cake. In another aspect, the Lactobacillus sp. bacteria in the disclosed composition may be provided in powdered form.

The Lactobacillus sp bacteria useful in the disclosed composition may be provided as a live culture, as a dormant material or a combination thereof. Those skilled in the art will appreciate that the Lactobacillus sp bacteria may be rendered dormant by, for example, a lyophilization process, as is well known to those skilled in the art.

An example of an appropriate lyophilization process may begin with a media carrying appropriate Lactobacillus sp bacteria to which, optionally, an amount of a phenolic compound may be added for cell protection prior to lyophilization. Examples of phenolic compounds may be provided as an extract from a natural plant source such as a berry (e.g. blueberry). In a further embodiment, an adjunct lyophilization protectant is combined with the Lactobacillus sp and phenolic compound prior to lyophilization. Examples of adjunct lyophilization protectants include, but are not limited to, distilled water, polyethylene glycol, sucrose, trehalose, skim milk, xylose, hemicellulose, pectin, amylose, amylopectin, xylan, arabinogalactan, starch (e.g., potato starch or rice starch) and polyvinylpyrrolidone. Gasses useful for the lyophilization process include but are not limited to nitrogen and carbon dioxide.

The disclosed compositions may be packaged in pharmaceutically acceptable vehicle, such as capsules, suppositories, tablets, food/drink and the like. Optionally, the disclosed Lactobacillus composition may include various pharmaceutically acceptable excipients, such as microcrystalline cellulose, mannitol, glucose, defatted milk powder, polyvinylpyrrolidone, starch and combinations thereof.

In one aspect, the disclosed compositions may be prepared as a capsule. The capsule (i.e., the carrier) may be a hollow, generally cylindrical capsule formed from various substances, such as gelatin, cellulose, carbohydrate or the like. The capsule may receive the Lactobacillus composition. Optionally, and in addition to the appropriate Lactobacillus composition, the capsule may include but is not limited to coloring, flavoring, rice or other starch, glycerin, caramel color and/or titanium dioxide.

In a specific embodiment, the capsule is engineered to protects its contents in a subject's stomach and release its contents (e.g. Lactobacillus composition) at the duodenum of a subject. In a specific example, the capsule pertains to duodenal release capsules, also referred to herein as DR capsules. Examples of DR capsules may have a shell comprised of a mixture of hypromellose or other hydrophilic cellulosic polymers mixed with an anionic polymer such as polyvinyl acetate, sodium carboxymethylcellulose, sodium alginate, xanthan gum, Carbopol (cross-linked acrylic acid polymers), cellulose acetate phthalate, hydroxypropyl-methylcellulose phthalate, methacrylic acid copolymer, hydroxypropylmethyl acetate succinate, and mixtures thereof. An exemplary version of DR capsules is AR-CAPS® acid resistant capsules (CAPSCANADA®, a Lyfe Group Co, Dania Beach, Florida). The capsule retains its stability against the pH levels of the stomach, but breaks down when it reaches the higher pH levels of the small intestines. See US Pat. Pub Nos. US20070048377 and US20180200192, and PCT Pub. WO2017/171535, which are incorporated herein in their entirety. In one example, the Lactobacillus composition is lyophilized and disposed within a DR capsule, such as of the type AR-CAPS®.

In another aspect, the Lactobacillus composition may be prepared as a suppository. The suppository may include but is not limited to the appropriate Lactobacillus composition, and one or more carriers, such as polyethylene glycol, acacia, acetylated monoglycerides, camuba wax, cellulose acetate phthalate, com starch, dibutyl phthalate, docusate sodium, gelatin, glycerin, iron oxides, kaolin, lactose, magnesium stearate, methyl paraben, pharmaceutical glaze, povidone, propyl paraben, sodium benzoate, sorbitan monoleate, sucrose talc, titanium dioxide, white wax and coloring agents.

In a further aspect, the Lactobacillus composition may be prepared as a tablet. The tablet may include the appropriate Lactobacillus composition and one or more tableting agents (i.e., carriers), such as dibasic calcium phosphate, stearic acid, croscarmellose, silica, cellulose and cellulose coating. The tablets may be formed using a direct compression process, though those skilled in the art will appreciate that various techniques may be used to form the tablets.

In yet another aspect, the disclosed composition may be formed as food or drink or, alternatively, as an additive to food or drink, wherein an appropriate quantity of Lactobacillus composition, is added to the food or drink to render the food or drink the carrier.

The concentration of the Lactobacillus sp. bacteria in the disclosed composition may vary depending upon the desired result, the type of bacteria used, the form and method of administration, among other things. For example, a composition may be prepared having a count of Lactobacillus sp. bacteria in the preparation of no less than about IxIO⁶ colony forming units per gram, based upon the total weight of the preparation.

Alternatively, one or more other excipients may be included in the composition to (1) impart satisfactory processing and compression characteristics to the composition (e.g., adjust the flowability, cohesion and other characteristics of the composition) and (2) give additional desirable physical characteristics to the tables (e.g. color, stability, hardness, disintegration). Mostly the excipients aid in the delayed release of the drug from the composition to achieve regional delivery to the lower GI. As used herein, the term “excipient” may include all excipients present in the dosage form, including all components other than the drug entity and the hydrocolloid gum from higher plants. A plurality of excipient substances may be present in any dosage form, and may include multiple substances having similar pharmaceutical function (e.g., lubricants, binders, diluents) or similar structure (e.g., a mixture of monosaccharides). Preferably the fewer excipients present the better. Such excipients are present in an amount sufficient to provide the composition with the desired delayed release/regional delivery characteristics, hardness rating and handling characteristics and will generally be present at a level of about 2% by weight to about 50% by weight, preferably about 2% by weight to about 40% by weight and more preferably about 2% to about 10% by weight. Excipients may be selected from many categories known in the pharmaceutical arts. The excipients used will be chosen to achieve the desired object of the invention keeping in mind the activity of the drug being used, as well as its physical and chemical characteristics such as water solubility and possible interactions with the excipients to be used. For example, with drugs that are more water soluble, generally a lower percentage by weight of excipients will be used, i.e., less than about 20% or from about 2% to about 15% by weight, preferably no more than about 10% by wt, while for drugs that are less water soluble a higher percentage by weight may be used, e.g., about 20% up to about 40% by wt. These levels may be adjusted to achieve the desired hardness and porosity of the final tablet composition to obtain the delayed release profile.

From the foregoing discussion, it is seen that one aspect of this invention is a particle mass of a solid dosage form that can be administered orally as a tablet. Thus, the composition is neither a liquid nor a gas, but a solid tablet having an amount of drug as a unit dosage. Generally, this unit dosage will be an amount that can be swallowed by a human subject and may vary from a total of about 100 milligrams to about 1500 mg, preferably no more than about 1200 mg and particularly no more than about 800 mg. For children, the size of the tablet may be significantly less than for adults, and for elderly patients who have difficulty swallowing, the total amount may be less than what would be viewed as a normal amount for adults. It is to be understood that the tablets of this invention may be designed as a single tablet having a unit dosage amount or several smaller tablets, e.g. 2-5, may be combined in a capsule for oral administration. The composition used to prepare the tablet may be granulated.

In a typical embodiment, the composition is formulated in accordance with routine procedures as a pharmaceutical composition adapted for oral administration to humans.

The amount of the Lactobacillus sp. which will be effective in the treatment of a particular disease or disorder will depend on the nature of the disease or disorder, and can be determined by standard clinical techniques. In addition, in vitro and in vivo assays may optionally be employed to help identify optimal dosage ranges. The dosage will depend on the body weight of the subject. However, in one example suitable dosage ranges for oral administration or parenteral administration may be about 10 μg to 100 mg, 20 μg to 50 mg, 0.1 mg to 20 mg, or 0.5 mg to 10 mg (calculated either per kg body weight or as total dose per individual). Suitable dosage ranges for intranasal administration are generally about 0.01 μg/kg body weight to 1 mg/kg body weight. Effective doses may be extrapolated from dose-response curves derived from in vitro or animal model test systems. Suppositories generally contain an active ingredient in the range of 0.5% to 10% by weight; oral formulations preferably contain 10% to 95% active ingredient. The general approaches to delivering drugs to the lower GI tract (e.g. small intestine and colon) for interaction with immune cells in the mucosa of the lower GI tract include: 1) enteric coating designed to release drug in the more alkaline environment of the gastrointestinal tract, 2) bio erodible coatings and matrices, 3) prodrugs, 4) timed-release systems and, 5) enteric polymeric material-based release systems that release drug after they transit through the stomach and reach the intestines. A general discussion of these approaches and others may be found in PCT Patent application No. PCT/US91/03G14 by Sintov and Rubinstein.

### Process of Preparation

Capsules containing Lactobacillus sp. can be prepared according to known techniques. See for example US Pat. Pub. 20170368049 incorporated by reference. In a specific embodiment, a Lactobacillus sp is first mixed with a pH reducing agent such as D-lactic acid, L-lactic acid, or mixture thereof, or acetic acid for a 1-5 hours, typically 1-2 hours. Typically, Lactobacillus sp cells are mixed with a pH reducing agent when they are in a stationary growth phase (e.g. phase where cell division has stopped). The concentration of the acid is between 3 and 6 g/L. The pH of the cell media is decreased with the pH reducing agent under conditions to promote the cells to trigger an acid tolerance response. The acid tolerance response confers resistance to various forms of stress, including freezing, lyophilization, and low pH environments. Following exposure to the reduced pH environment, the media is removed by centrifugation and mixed with a carrier, such as non-fat skim milk or berry mixture as is described elsewhere herein, and then lyophilized. The lyophilized mixture of cells and carrier is then disposed within a DR capsule. In a specific embodiment, the DR capsule contains a trace amount of the pH reducing agent (between 2 and 4 g/L).

For preparing solid compositions such as tablets, the principal active ingredient may be mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the intestine or lower GI tract, or to otherwise be delayed in release. A variety of materials can be used for such enteric layers or coatings, such materials including a number of polymeric acids and mixtures of polymeric acids with such materials as shellac, acetyl alcohol and cellulose acetate.

In preparing the tablet compositions of this invention one may use pharmaceutical compression or molding techniques, preferably the former due to its adaptability to large scale production methods. Using techniques known in the art, the tablets of the invention may take any appropriate shape such as discoid, round, oval, oblong, cylindrical, triangular, hexagonal, and the like. The tablets may be coated or uncoated. If coated they may be sugar-coated (to cover objectionable tastes or odors and to protect against oxidation), film coated (a thin film of water-soluble matter for similar purposes), or enteric coated (to resist dissolution in gastric fluid but allow disintegration of the coating in the small intestine—as discussed herein before). Depending on whether the tablet is a uniform matrix tablet, an active core tablet or a concentration gradient tablet, the process for preparation will vary slightly.

In order to ensure tablet hardness and uniformity of weight, content and other items, it is preferable to prepare the tablets having the composition of this invention by using a pre granulation technique. In general, the granulation techniques can include the wet granulation method, the fluid bed granulation method, the dry granulation method or direct compression.

Once the tablets are appropriately formed, they can then be coated by any of the necessary coating techniques as discussed in Chapter 90 of Remington's. For example, the tablets may be sugar-coated in accordance with the procedure discussed therein or film coated or preferably enterically coated. Enteric coating is preferred in the tablets of this invention to minimize the release of any of the drug in the upper GI and assure the release to the lower GI particularly the colon. As much as pertinent of the Remington's sections of Chapters 88 and 90 is incorporated herein by reference.

### Administration

In some embodiments, the composition embodiments comprising Lactobacillus sp. described herein will be administered orally to a mammalian subject in need thereof using a level of pharmaceutical composition that is sufficient to provide the desired physiological effect. The mammalian subject may be a domestic animal or pet but preferably is a human subject. The level of pharmaceutical composition needed to give the desired physiological result is readily determined by one of ordinary skill in the art. Other parameters that may be taken into account in determining dosage for the pharmaceutical composition embodiments described herein may include disease state of the subject or age of the subject.

The compositions may take the form of suspensions, solutions or emulsions in oily or aqueous vehicles and may contain formulatory agents such as suspending, stabilizing and/or dispersing agents. In some embodiments, the composition embodiments described herein may be administered orally or intravenously via parenteral nutritional therapy to a subject via an emulsion. The emulsion may include, in some embodiments, an aqueous continuous phase and a dispersed phase. The boundary between the phases called the “interface”. The present emulsions are adapted for application to a mucosal surface of a vertebrate animal, preferably a mammal, including humans. These compositions improve the permeability and bioavailability of active compounds after application to a mucous surface. Mucosal surfaces of interest include the intestinal mucosa. Use of bioadhesive polymers in pharmaceutical emulsions affords enhanced delivery of drugs in bioadhesive polymer-coated suspensions, in some examples. Bioadhesive pharmaceutical emulsions may be used to deliver the Lactobacillus sp. described herein to: a) prolong the residence time in situ, thereby decreasing the number of drug administrations required per day; and b) may be localized in the specified region to improve and enhance targeting and bioavailability of delivered drugs.

The ability to retain and localize a drug delivery emulsion in a selected region leads to improved bioavailability, especially for drugs exhibiting a narrow window of adsorption due to rapid metabolic turnover or quick excretion. Intimate contact with the target absorption membrane improves both the extent and rate of drug absorption.

Bioadhesion is the characteristic of certain natural and synthetic polymers of binding to various biological tissues. Of particular interest are polymers which bind to the mucous lining that covers the surface of many tissues which communicate directly or indirectly with the external environment, such as the gut, for example. Mucus binding polymers may be referred to as mucoadhesive. Several bioadhesive, and specifically mucoadhesive, polymers are known. The chemical properties of the main mucoadhesive polymers are summarized as follows:
a. strong H-bonding groups (—OH, —COOH) in relatively high concentration;
b. strong anionic charges;
c. sufficient flexibility of polymer backbone to penetrate the mucus network or tissue crevices;
d. surface tension characteristics suitable for wetting mucus and mucosal tissue surfaces; and
e. high molecular weight.

Bioadhesive polymers may be used in the pharmaceutical composition embodiments described herein, examples of bioadhesive polymers currently used in pharmaceutical preparations include: carboxymethylcellulose (CMC), hydroxypropylmethylcellulose (HPMC), polyacrylic and polymethacrylic acid and their derivatives, pectin, alginic acid, chitosan, polyvinylpyrrolidone, hyaluronic acid, and polyvinyl alcohol. The most frequently used polymer is Carbopol (Carbomer), which is a high molecular weight polyacrylic acid polymer. It is used in many formulations for bioadhesive drug delivery systems, as a suspending agent, as a tablet coating, and in ocular suspensions.

Pharmaceutical composition embodiments described herein may include the Lactobacillus composition incorporated into inert lipid carriers such as oils, surfactant dispersions, emulsions, liposomes etc. Self-emulsifying formulations are ideally isotropic mixtures of oils, surfactants and co-solvents that emulsify to form fine oil in water emulsions when introduced in aqueous media. Fine oil droplets would pass rapidly from stomach and promote wide distribution of drug throughout the GI tract, thereby overcome the slow dissolution step typically observed with solid dosage forms. These embodiments may provide control release self-emulsifying pellets, microspheres, tablets, capsules etc. that increase the use of “self-emulsification.”.

In further embodiments, composition embodiments described herein may be microencapsulated for delivery to a subject.

Microencapsulation (ME) offers the potential to reduce the adverse effects on probiotic viability in the gastrointestinal (GI) tract environment. ME separates microorganism cells from their environment until they are released. Controlled release of the Lactobacillus is a particular benefit of ME. It is beneficial for encapsulated probiotic microorganisms to be released in the small intestine where the Peyer's patches exist to activate the immune system, in some embodiments.

Oral delivery will be the most straightforward mode of administration to deliver the Lactobacillus sp. to the gut mucosa. However, in alternative embodiments, other methods of administration are contemplated. Accordingly, suitable methods for administering a Lactobacillus composition in accordance with the methods of the presently-disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, dermally (e.g., topical application), intratracheal installation, surgical implantation, transdermal delivery, local injection, intranasal delivery, and hyper-velocity injection/bombardment. Where applicable, continuous infusion can enhance drug accumulation at a target site (see, e.g., U.S. Pat. No. 6,180,082). In some embodiments of the therapeutic methods described herein, the therapeutic compositions are administered orally, intravenously, intranasally, or intraperitoneally to thereby treat a disease or disorder.

Regardless of the route of administration, the compositions of the presently-disclosed subject matter typically not only include an effective amount of a Lactobacillus sp. but are typically administered in amount effective to achieve the desired response. As such, the term “effective amount” is used herein to refer to an amount of the therapeutic composition (e.g., a Lactobacillus sp., and a pharmaceutically vehicle, carrier, or excipient) sufficient to produce a measurable biological response (e.g., a decrease in diabetes symptoms or increase in immune function). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

### EXAMPLES

### Example 1: Materials and Methods

### Bacterial Growth

L. johnsonii N6.2 was grown de Man, Rogosa, Sharpe media (MRS) media at 37° C. under static condition as previously described (Harrison et al., 2021; Valladares et al., 2010).

### Animal Model and Intervention Details

This study was carried out in male homozygous db/db mice strain 000697 (N=6 animals per group) purchased from Jackson Laboratory (Bar Harbor, ME). Feeding was started at 4 weeks of age and continued for 14 weeks (end point, animals were 20 weeks old).

At 4 weeks, mice were provided with drinking water containing sulfamethoxazole/trimethoprim solution at a final concentration of 40 mg/ml sulphamethoxazole/8 mg/ml trimethoprim for a total of 3 days followed by a 3 day washout period to reduce normal oral flora and facilitate the colonization of the murine oral cavity.

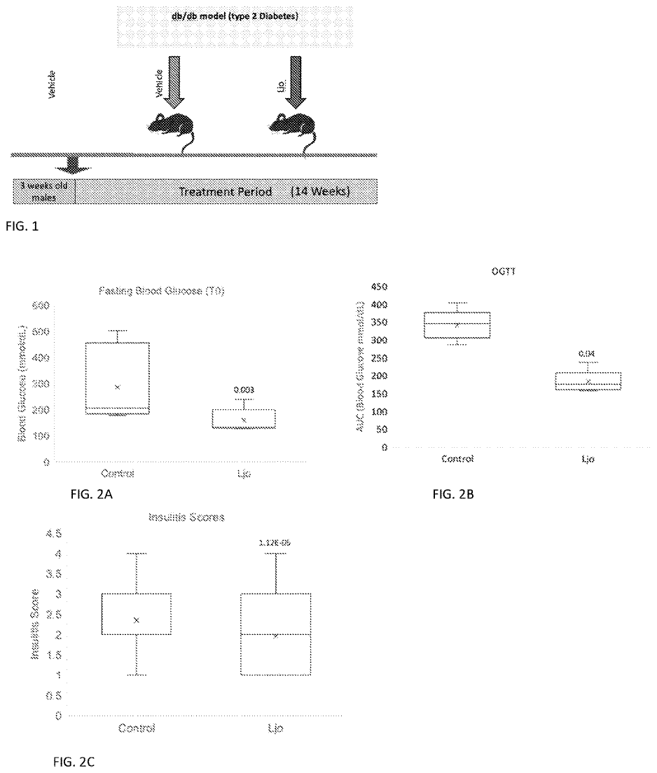

Beginning at 4 weeks post-antibiotic washout through week 20, mice were fed 100 μl L. johnsonii N6.2 at a concentration of 10e9 resuspended in 1×PBS media three times per week (Monday, Wednesday, Friday). During each feeding, urine and feces were also routinely collected while handling each animal in a separate container. As a vehicle control, animals were fed 100 μl 1×PBS media three times per week (Monday, Wednesday, Friday). Animals were weighed weekly. A diagram of the study design is provided in FIG. 1.

Endpoint criteria included >=15% weight loss from baseline, body condition score of 2 or less, inability to reach food or water, impaired mobility beyond experimental endpoint, tumors, labored breathing, respiratory distress, cyanosis, tremors, convulsions, or seizures lasting more than 1 minute or occurring more than once per day, dehydration over 24 hours, and moribund. All animals reached experimental endpoint of 16 weeks post initial infection at which point they were euthanized primarily by inhalation of carbon dioxide at a dosage of 30-70% chamber volume per minute and secondarily by cervical dislocation.

### Blood Collection

Blood samples were collected following the procedure listed in Ayala et al. (2010). Briefly, blood was collected from the tail tip by cutting 1-2 mm of tissue from the tail tip distal to the bone with sharp scissors or a scalpel, then obtaining blood by direct flow or gently massaging the tail. Blood was collected in a capillary tube or other containing. Following the initial cut, a 2-hour recovery period was allowed prior to collecting the first sample. Subsequent samples were obtained by gently removing the scab on the tail and repeating the massaging procedure.

### Fasting Blood Glucose and Oral Glucose Tolerance Test

Mice were fasted for 8 h prior to testing at the beginning of week 16. Tails were previously cut by the above procedure for blood collection, and glucose was measured by removing the scab, discarding the first drop of blood, and using the second drop of blood (<5 μl) for detection of blood glucose using a glucometer (model). Following fasting, oral glucose tolerance tests were carried out using 2 g/kg body weight glucose (20% solution) fed orally using a sterile pipette. Tail blood samples (<5 μl) were collected by removing tail scab and gently massaging the tail as previously described at 0, 30, 60, 90, and 120 minutes post-glucose loading. Blood glucose was detected via a glucometer (model) as described above. The area under the curve (AUC) was determined utilizing the timepoints from 0-120 minutes after glucose administration.

### Histology Analysis

All tissues were collected and fixed overnight (12 hours) in formalin, washed twice in 1×PBS, then stored in 1×PBS at 20° C. until further analysis. Fixed tissues were then submitted to University of Florida's Molecular Pathology Core for hematoxylin and eosin (H & E) staining (add methodology from path core here).

### Insulitis Scoring

One H & E stained slide of pancreatic tissue for each of the 24 animals were first blinded by a single lab member, removing identifying information for scoring lab members. Two lab members were assigned to score each slide for insulitis following protocols modified from Efrat et al. (2001) and Li et al. (2002). Briefly, slides were visualized at 10× and 100×, islet cells were enumerated, then visually analyzed and scored from 1-4 based upon percent infiltration of immune cells. A score of 1 reflected a little to no infiltration of immune cells between 0-20% infiltration, a score of 2 reflected minor infiltration from 20-50% infiltration, a score of 3 reflected major infiltration from >50%, <100% infiltration, and a score of 4 reflected near-total or total infiltration of islet cells by immune cells. Again, percentages and the respective scores were estimated solely based upon visual assessment and were independently scored by two lab members. After scoring, slides were unblinded and analyzed by average insulitis score.

### Steatosis Scoring

One H & E stained slide of liver tissue for each of 24 animals were blinded by a single lab member, removing identifying information for the lab member scoring the slides. The slides were visualized on a 10× field and five photos per slide were taken at random portions of the tissue. Steatosis was enumerated following methods modified from Takahashi et al. (2014). Briefly, each of the five photos per animal were then analyzed in image-J, measuring the total area of the image and the area of tissue with visible steatosis using the scale bar imposed over the image for scaling. The area with steatosis and the total area were then converted to percent area with steatosis and assigned a score between 1 and 4. A score of 1 reflected <5% area with steatosis, a score of 2 represented 5%-33% area with steatosis, a score of 3 represented 33%-66% steatosis, and a score of 4 represented >66% area with steatosis. Images were also visually analyzed and assigned a score from 1-3 for lobular inflammation, with a score of 1 representing no foci of inflammatory cells, a score of 2 representing <2 foci of inflammatory cells, and a score of 3 representing >2 foci of inflammatory cells. Images were also assessed for ballooning cells and scored between 1 and 3, with a score of 1 representing no ballooning cells, a score of 2 representing few ballooning cells, and a score of 3 representing many ballooning cells. Each of these scores from five images were then unblinded and averaged per animal and compared between the four groups.

### Adipocyte Analysis

Visceral adipose was collected from the left perirenal area of all mice. Subcutaneous adipose collected from the right side of all mice. Tissues were harvested and fixed in formalin as described above, then stained with H & E as described. H & E stained slides were blinded by a single lab member for the scoring lab member who then analyzed the slides by microscopy on a 10× field. Five images per slide were taken at random areas of the tissue for analysis in Image-J. In Image-J, the adipocytes were enumerated and their diameter and area measured using the scale bar imposed over the image for scale. The images were then unblinded and the number of adipocytes, their diameter, and area were each averaged and compared among the four groups.

### Kidney Nephropathy Scoring

One H & E stained slide of kidney tissue for each of 24 mice were first blinded by a single lab member for a separate scoring lab member. Slides were visualed by microscopy on a 40× field, and images of at least five separate glomeruli were taken by the lab member. To assess diabetic nephropathy, images were analyzed in Image-J measuring the area of the glomerulus, bowman's capsule, immune cells, and the glomerular matrix. Percentages of cells per glomerulus and the percent glomerular matrix were calculated for each image. Cell infiltrates were also enumerated for each image. After unblinding images, the scores were then averaged per animal and compared between groups.

### RNA Isolation and Quantification

RNA was isolated via trizol method. Briefly, around 50 to 100 mg of frozen liver were placed in an Eppendorf tube containing 300 ul of trizol (Tri reagent, ZYMO Research, Irvine, CA). The tissue was homogenized using a small sample mixer and homogenizer. To remove cell debris, tubes were centrifuge at 12,000×g for 5 minutes. The supernatant was transferred to an RNA purification spin column (zymo research, Irvine, CA). RNA isolation was proceeded according to the manufacturer's protocol. DNA decontamination was done via DNase digestion (QIAGEN, Germantown, MD) according to the manufacturer's protocol. After DNase treatment, RNA quality was monitored on 1% agarose gels, and RNA quantification was checked using Thermo Scientific Nanodrop One Microvolume UV-vis spectrophotometer (Thermo Fisher Scientific, Grand Island, NY).

### RNAseq Analysis of Liver Tissue

The degradation and contamination of the RNA were monitored on 1% agarose gels. RNA purity was checked using the NanoPhotometer® spectrophotometer (IMPLEN, CA, USA). RNA integrity and quantitation were assessed using the RNA Nano 6000 Assay Kit of the Bioanalyzer 2100 system (Agilent Technologies, CA, USA). To prepare the library for transcriptome sequencing, 1 μg of RNA per sample was used as input material for the RNA sample preparations. Sequencing libraries were generated using NEBNext® Ultra™ RNA Library Prep Kit for Illumina® (NEB, USA) following manufacturer's protocol and index codes were added to attribute sequences to each sample. Briefly, mRNA was purified from total RNA using poly-T oligo-attached magnetic beads. Fragmentation was carried out using divalent cations under elevated temperature in NEBNext First Strand Synthesis Reaction Buffer (5×). First strand cDNA was synthesized using random hexamer primer and M-MuLV Reverse Transcriptase (RNase H−). Second strand cDNA synthesis was subsequently performed using DNA Polymerase I and RNase H. Remaining overhangs were converted into blunt ends via exonuclease/polymerase activities. After adenylation of 3′ ends of DNA fragments, NEBNext Adaptor with hairpin loop structure were ligated to prepare for hybridization. To select cDNA fragments of preferentially 150~200 bp in length, the library fragments were purified with AMPure XP system (Beckman Coulter, Beverly, USA). Then 3 μl USER Enzyme (NEB, USA) was used with size-selected, adapter-ligated cDNA at 37° C. for 15 min followed by 5 min at 95° C. before PCR. Then PCR was performed with Phusion High-Fidelity DNA polymerase, Universal PCR primers and Index (X) Primer. At last, PCR products were purified (AMPure XP system) and library quality was assessed on the Agilent Bioanalyzer 2100 system. The clustering of the index-coded samples was performed on a cBot Cluster Generation System using PE Cluster Kit cBot-HS (Illumina) according to the manufacturer's instructions. After cluster generation, the library preparations were sequenced on an Illumina platform and paired-end reads were generated. FastQC was used to filter high quality reads. The data was analyzed using Kallisto v0.46.1 (Bray 2016). In order to create an index and map the high-quality raw reads. EnsDb.Mmusculus.v79 package in RStudio (Rainer J 2017, R Team 2020) was used as a reference transcriptome for mRNA to create an index and map the high-quality raw reads. Then, the differential abundance of mRNA transcripts was evaluated using limma 3.52.4, edgeR 3.38.4 and SVA 3.44.0 packages in RStudio (Smyth 2002, Robinson 2010, Leek J T 2022). All the graphics were generated using ggplot2 3.3.6 R package (Wickham 2009).

DNA Extraction, 16S rRNA Sequencing and Microbiota Analysis

DNA was extracted from stools samples using PowerFecal DNA isolation kit (MO BIO Laboratories) following manufacturer instructions. Quantification of DNA was performed using a Nanodrop spectrophotometer (Thermo Scientific, Wilmington, DE). Libraries were constructed using V4 universal primers were used for 16S rRNA amplification and subsequent Illumina MiSeq sequencing at the ICBR core facility at UF (Caporaso 2012). Sequencing was performed on an Illumina MiSeq with a 300-bp paired-end protocol, using single indexing. Sequencing reads were parsed by Illumina index at the sequencing center. Paired reads were merged, and primers and adaptors were removed using trimmomatic (Bolger 2014), and illumina-utils (Eren 2013). Reads were processed using QIIME2 v2022.2, with a sample read depth range from 28,234 to 42,246 reads per sample, and an average of 33,639 reads (Bolyen 2019). The Silva reference database version 138 (Quast 2013) was used for the replicon sequence variant table construction and the taxonomy assignment was performed with DADA2 (Callahan 2016). Community structure was analyzed in R with phyloseq (McMurdie 2013) and plotted with ggplot2 (Wickham 2009). Analysis of similarities (ANOSIM) was performed in R using VEGAN v2.6-2 (Oksanen 2020). α diversity metrics expressed as Shannon index between groups were compared using the Kruskal-Wallis or Wilcoxon test for comparison of two independent groups only. Significance was set at P≤0.05. Differences in Bray-Curtis dissimilarities between groups were calculated using the adonis2 function from the vegan R package (900 permutations) (Oksanen 2020), β diversity was represented using nonmetric multidimensional scaling (NMDS) with a stress level of 0.08 for two dimensions.

### Example 2: L. johnsonii N6.2 Administration Decrease the Levels of Fasting Glucose in the DB/DB Model of T2D

The effect of L. johnsonii N6.2 in T2D was evaluated through supplementation into the DB/DB model of T2D diabetes. To this end 4 weeks old males were obtained from Jackson Laboratory. L. johnsonii was orally fed at 10⁹ CFU three times a week while the control group was feed with PBS for 14 weeks. No significant changes in weight were observed between the groups. The role of L. johnsonii on glucose tolerance was evaluated after 13 weeks of treatment. It was found that the Ljo administration resulted a significant decrease in the fasting glucose levels as well as a decrease in the area under the curve (AUC) in the glucose tolerance test (OGTT) (FIGS. 2A and 2B). In agreement with these results, we found that the histological analyses of the pancreas showed a significant decrease in the insulitis scores in the Ljo fed group (FIG. 2C).

### Example 3: L. johnsonii N6.2 Administration Decrease the Steatosis Scores

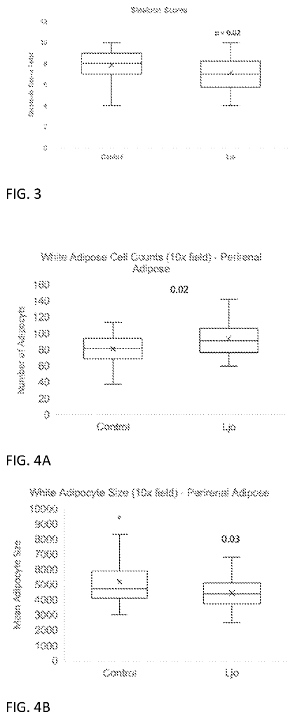

To investigate the effect of L. johnsonii N6.2 on the liver, liver tissue was assessed for diabetic steatosis pathology by scoring for cells affected by steatosis, inflammation, and ballooning. Scores were summed for a total steatosis score, averaged, and compared between groups with greater total scores being indicative of more advanced diabetic steatosis of the liver. Comparison of the total steatosis scores shows significantly lower (p<0.05) scores in DB/DB mice fed L. johnsonii N6.2 compared to controls (FIG. 3).

### Example 4: L. johnsonii N6.2 Administration Increase the Number of White Adipose Cells while Decreasing its Size

Since steatosis has been linked to adipocyte hypertrophy, we assessed the perirenal white adipose cell numbers and size. 5 Images/fields were analyzed per slide/animal. A beneficial effect is associated with an increase in the number of cells and a decrease in the size and diameter of the cells. FIG. 4A shows that the number of white adipose cells significantly increase (p<0.05) in the L. johnsonii orally treated DB/DB model. FIG. 4B shows that the size of white adipose cells significantly decreases (p<0.05) in the L. johnsonii orally treated DB/DB model. FIG. 4C shows that the mean diameter of white adipose cells decreases in the L. johnsonii orally treated DB/DB model.

### Example 5: L. johnsonii N6.2 Administration Decrease Kidney Nephropathy by Decreasing the Size of the Mesangliar Matrix

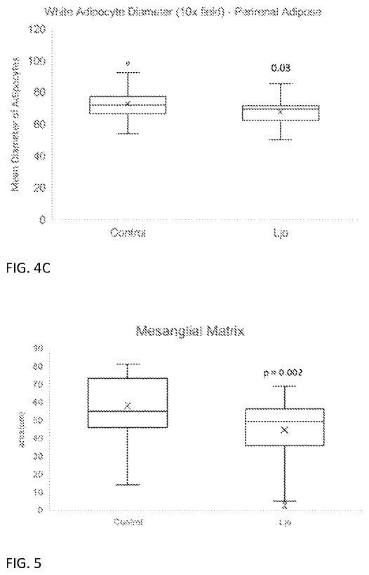

In the DB/DB mouse model, as result of hyperglycemia a progressive expansion of the kidneys mesangial matrix is observed. While the average size of the glomeruli did not differ between control and L. johnsonii N6.2 fed mice, we observed that the administration of L. johnsonii N6.2 resulted in a significant decrease (p<0.05) in the percent mesangliar matrix (FIG. 5). The observed decrease in mesanglial expansion is in agreement with the decrease in hyperglycemia observed upon administration of L. johnsonii N6.2. The modification of all these parameters is an indicator of a beneficial effect of L. johnsonii N6.2 supplementation.

According to these results, L. johnsonii N6.2 has a beneficial effect on DB/DB mice by decreasing the negative effects of T2D generally observed and compared with the controls.

### Example 6: L. johnsonii N6.2 Feedings Affect the Community Structure of the Gut Microbiome of the DB/DB Model

Using 16S rRNA sequencing of the V4 gene variable region, samples of stools from the end point of the experiment were evaluated to determine the microbiome composition. Despite that no significant differences were found in α diversity (P=0.4), β diversity analysis evidenced two different community structures when the animals feed with L. johnsonii N6.2 were compared with the controls. Using nonmetric multidimensional scaling (NMDS) based on Bray-Curtis distance, a strong cluster separation was elucidated between the treatment and the control group (R2=0.4, p<0.01, stress=0.08). Statistical differences where observed at phylum level (P<0.05), led by a decrease in Bacteriodota phylum in the treatment group, this difference was consistent with family and genus taxonomic levels, were the statistical differences were observed in family Muribacullaceae and unclassified genus. Significant differences were also observed between LJ and control group on genus Eubacterium xylanophilum group, with higher abundance in the treatment group (p<0.05).

### Example 7: L. johnsonii N6.2 Administration Induce Significant Changes in the Transcriptome of Liver Tissue

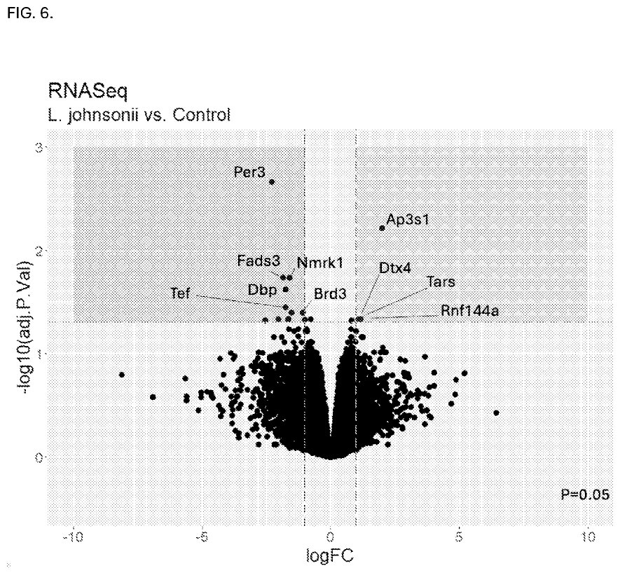

Global RNAseq analyses indicated significant changes in the transcriptome of liver tissue resulting from the administration of L. johnsonii N6.2. A significant decrease (padjust <0.05) was observed in the expression of Per3, Fads3, NmrK1, Tef, Dbp and Brd3 while the expression of Ap3s1, Dtx4, Tars and RNf144a was significantly induced (FIG. 6).

### REFERENCES

Bray N L, Pimentel H, Melsted P, Pachter L. Near-optimal probabilistic RNA-seq quantification. Nat Biotechnol. 2016 May; 34(5):525-7. doi: 10.1038/nbt.3519. Epub 2016 Apr. 4. Erratum in: Nat Biotechnol. 2016 Aug. 9; 34(8):888. PMID: 27043002.
Rainer J (2017). EnsDb.Mmusculus.v79: Ensembl based annotation package. R package version 2.99.0. DOI: 10.18129/B9.bioc.EnsDb.Mmusculus.v79
R Core Team (2020). R: A language and environment for statistical computing. R Foundation for Statistical Computing, Vienna, Austria. URL: https://www.T-project.org/.
Robinson M D, McCarthy D J, Smyth G K. edgeR: a Bioconductor package for differential expression analysis of digital gene expression data. Bioinformatics. 2010 Jan. 1; 26(1):139-40. doi: 10.1093/bioinformatics/btp616. Epub 2009 Nov. 11. PMID: 19910308; PMCID: PMC2796818.
Gordon K. Smyth, Matthew Ritchie, Natalie Thorne, James Wettenhall, Wei Shi and Yifang Hu (2002). Limma: Linear Models for Microarray and RNA-Seq Data User's Guide. Bioinformatics Division, The Walter and Eliza Hall Institute of Medical Research, Melbourne, Australia.
Leek J T, Johnson W E, Parker H S, Fertig E J, Jaffe A E, Zhang Y, Storey J D, Torres L C (2022). sva: Surrogate Variable Analysis. R package version 3.46.0.
Wickham, H. (2009) Ggplot2: Elegant Graphics for Data Analysis. 2nd Edition, Springer, New York. https://doi.org/10.1007/978-0-387-98141-3
Alpers C E, Hudkins K L. Pathology identifies glomerular treatment targets in diabetic nephropathy. Kidney Res Clin Pract. 2018 June; 37(2):106-111. doi: 10.23876/j.krcp.2018.37.2.106. Epub 2018 Jun. 30. PMID: 29971205; PMCID: PMC6027807.
Pourghasem M, Shafi H, Babazadeh Z. Histological changes of kidney in diabetic nephropathy. Caspian J Intern Med. 2015 Summer; 6(3):120-7. PMID: 26644877; PMCID: PMC4650785.
Takahashi Y, Fukusato T. Histopathology of nonalcoholic fatty liver disease/nonalcoholic steatohepatitis. World J Gastroenterol. 2014 Nov. 14; 20(42):15539-48. doi: 10.3748/wjg.v20.i42.15539. PMID: 25400438; PMCID: PMC4229519.
Efrat S, Serreze D, Svetlanov A, Post C M, Johnson E A, Herold K, Horwitz M. Adenovirus early region 3 (E3) immunomodulatory genes decrease the incidence of autoimmune diabetes in NOD mice. Diabetes. 2001 May; 50(5):980-4. doi: 10.2337/diabetes.50.5.980. PMID: 11334441.
Li F, Filippova M, Fagoaga O, Nehlsen-Cannarella S, Escher A. Decreased insulitis and blood glucose levels after injection of GAD-transduced lymphocytes into NOD mice. Mol Ther. 2002 December; 6(6):701-9. doi: 10.1006/mthe.2002.0801. PMID: 12498766.
Ayala J E, Samuel V T, Morton G J, Obici S, Croniger C M, Shulman G I, Wasserman D H, McGuinness O P; NIH Mouse Metabolic Phenotyping Center Consortium. Standard operating procedures for describing and performing metabolic tests of glucose homeostasis in mice. Dis Model Mech. 2010 September-October; 3(9-10):525-34. doi: 10.1242/dmm.006239. Epub 2010 Aug. 16. PMID: 20713647; PMCID: PMC2938392.

What is claimed is:

1. A method for treating type 2 diabetes (T2D) in a subject in need, wherein said method comprises administering to the subject in need an effective amount of a composition comprising a Lactobacillus sp wherein said composition, optionally, further comprises a carrier.

2. The method, according to claim 1, wherein the subject in need is in a pre-diabetic state.

3. The method, according to claim 1, wherein the composition is a DR capsule containing a lyophilized mixture of the Lactobacillus sp and the carrier, wherein the DR capsule comprises a shell that is, optionally, comprised of hydrophilic cellulose derivative and an anionic polymer.

4. The method, according to claim 3, wherein the carrier is non-fat skim milk, infant formulae, or a berry mixture, or a mixture of the foregoing.

5. The method, according to any of claims 1-4, wherein the composition further comprises an antioxidant.

6. The method, according to claim 3, wherein the hydrophilic cellulose derivative is hypromellose and the anionic polymer is selected from polyvinyl acetate phthalate, sodium carboxymethylcellulose, sodium alginate, xanthan gum, Carbopol (cross-linked acrylic acid polymers), cellulose acetate phthalate, hydroxypropyl-methylcellulose phthalate, methacrylic acid copolymer, hydroxyppropylmethyl acetate succinate, and mixtures thereof.

7. The method, according to claim 1, wherein the composition further comprises vitamins, minerals, peptides, enzymes, and coenzymes.

8. The method, according to claim 1, wherein the Lactobacillus sp is administered to the subject in need at a dosage ranging from 10² to 10¹¹ Lactobacillus per day.

9. The method, according to claim 1, wherein the composition comprises about 10² to 10¹¹ Lactobacillus johnsonii.

10. The method, according to any of claims 1, 3-9, 13 and 14, wherein the subject in need has T2D.

11. The method, according to any of claims 1, 3-9, 13 and 14, wherein the subject in need is at risk for T2D.

12. The method, according to claim 1, wherein the composition is administered prior to the appearance of clinical symptoms of T2D.

13. The method according to any of claims 1-12, wherein the Lactobacillus sp comprises, consists essentially of or consists of Lactobacillus johnsonii N6.2 having ATCC Accession No. PTA-122064.

14. The method of any of claims 1-13, wherein the composition further comprises a phenol.

15. A capsule, optionally a DR capsule, comprising a lyophilized amount of a Lactobacillus sp and a carrier contained within the capsule, wherein the capsule comprises a shell that is, optionally, comprised of hydrophilic cellulose derivative and an anionic polymer.

16. The capsule of claim 15, wherein the carrier is non-fat skim milk, infant formulae, or a berry mixture, or a mixture of the foregoing.

17. The capsule of claim 15 or claim 16, wherein the composition further comprises an antioxidant.

18. The capsule of claim 15, wherein the hydrophilic cellulose derivative is hypromellose and the anionic polymer is selected from polyvinyl acetate phthalate, sodium carboxymethylcellulose, sodium alginate, xanthan gum, Carbopol (cross-linked acrylic acid polymers), cellulose acetate phthalate, hydroxypropyl-methylcellulose phthalate, methacrylic acid copolymer, hydroxyppropylmethyl acetate succinate, and mixtures thereof.

19. The capsule of claim 15, further comprising vitamins, minerals, peptides, enzymes, and coenzymes contained with the DR capsule.

20. The capsule of claim 15, further comprising D-lactic acid, L-lactic acid, a mixture of D-lactic acid and L-lactic acid or acetic acid, or a mixture thereof.

21. A method for alleviating type 2 diabetes (T2D) in a subject in need, wherein said method comprises administering to the subject in need a capsule according to any of claims 15-20.

22. A method of treating fatty liver disease in a subject in need, wherein said method comprises administering to the subject in need an effective amount of a composition comprising a Lactobacillus sp wherein said composition, optionally, further comprises a carrier.

23. The method of claim 22, wherein the Lactobacillus sp is Lactobacillus johnsonii N6.2 having ATCC Accession No. PTA-122064.

24. The method of claim 22 or 23, wherein the composition is a form of a capsule according to any of claims 15-20.

25. A method of treating diabetic nephropathy in a subject in need, wherein said method comprises administering to the subject in need an effective amount of a composition comprising a Lactobacillus sp wherein said composition, optionally, further comprises a carrier.

26. The method of claim 25, wherein the Lactobacillus sp is Lactobacillus johnsonii N6.2 having ATCC Accession No. PTA-122064.

27. The method of claim 25 or 26, wherein the composition is a form of a capsule according to any of claims 15-20.

28. The method of any of claims 25-27, wherein the subject in need has early diabetic nephropathy.

29. The method of any of claims 25-27, wherein the subject in need has overt diabetic nephropathy.

30. The method of any of claims 1-14 and 22-29, wherein the Lactobacillus sp is isolated.
